(19) United States
(12) Patent Application Publication
(10) Pub. No.: US 2009/0238893 A1
(43) Pub. Date: Sep. 24, 2009

(54) NUTRITIONAL FORMULATION

(76) Inventors: Jane Elizabeth Langford, Liverpool (GB); Ian Sullivan, Moreton (GB); Catherine Teresa Deering, Liverpool (GB); Sandra Helen Giffen, Liverpool (GB)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 12/306,544

(22) PCT Filed: Jun. 27, 2007
(86) PCT No.: PCT/GB07/02406

§ 371 Date: Dec. 24, 2008

(30) Foreign Application Priority Data
Jun. 26, 2006 (GB) 0612671.8

(51) Int. Cl. A61K 33/42

(52) U.S. Cl. 424/602

(57) ABSTRACT

A nutritional formulation for the treatment of inflammatory conditions of the gastrointestinal tract (particularly Crohn's Disease in paediatric patients) comprises: (a) a lipid source, (b) a carbohydrate source, and (c) a protein source. wherein the lipid source comprises a weight ratio of n-6:n-3 fatty acids of 1:1 to 3:1, at least 90% by weight of the n-3 fatty acids in the lipid source are provided by α-linolenic acid and at least 90% by weight of the n-6 fatty acids in the lipid source are provided by linoleic acid and components (a), (b) and (c) together provide at least 400 kcal of energy per 100 g total weight of (a)-(c).

The present invention relates to a nutritional formulation for the treatment of inflammatory conditions of the gastrointestinal (GI) tract. Examples of such inflammatory conditions include, but are not limited to, Inflammatory Bowel Disease (IBD) and gastrointestinal inflammation associated with cystic fibrosis (CF).

Inflammatory conditions of the GI tract cause considerable discomfort and inconvenience to sufferers. For example, mentioned Inflammatory Bowel Diseases such as Crohn's Disease (CD) and Ulcerative Colitis (UC) both have symptoms of abdominal pain and diarrhoea.

The pathology of Crohn's Disease is predominately characterised by inflamed sections of gut that may occur at any point throughout the gastrointestinal tract with apparently normal segments of gut in between. This inflammation is present throughout the entire thickness of the gut wall and can often result in the formation of fistulae. The disease consists of periods of acute inflammation (often referred to as “flare-up”) interspersed with periods of remission where the disease may remain quiescent for varying periods of time. In contrast to Crohn's disease ulcerative colitis only affects the large intestine, usually starting in the rectum and extending proximally. Unlike Crohn's disease which commonly shows skip lesions, ulcerative colitis is continuous, areas of normal gut are not found between lesions. Ulcerative colitis is a chronic relapsing inflammatory disorder, and diffuse superficial inflammation is seen in the large intestine. The mucosa is granular and haemorrhagic, rarely involving the muscle layer, unlike Crohn's is which deep fissure ulcers form.

Cystic fibrosis is manifestly a disease of the exocrine orgens and therefore affects respiratory and digestive tracts amongst others. Intestinal and respiratory inflammation with high concentrations of inflammatory markers in the blood of CF patients correlate with disease severity.

Both Crohn's Disease and Ulcerative Colitis occur frequently in the paediatric population (approximately 25% of cases are paediatric) with a peak incidence in the second decade of life. Monitoring of paediatric Crohn's Disease within the UK has shown that the incidence has more than doubled over the last twenty years.

Various drug therapies are available for treatment of IBD conditions. Such therapies include the use of corticosteroids. These are most often administered during acute disease and are effective in producing temporary remission from symptoms. However, there are associated side effects with the use of these drugs for treatment of the paediatric population as they can result in a reduction in growth during puberty and a decrease in bone mineral density, resulting ultimately in increased incidence of osteopaenia and osteoporosis in this population. Other drugs such as aminosalicylates (5-ASAs) are available for use in the paediatric population. 5-ASA's also have some associated side effects ranging from relatively mild (nausea, vomiting, diarrhoea and headache) to potentially severe, but rare, effects such as inflammation of major organs such as lungs heart and pancreas.

Another treatment option is surgery. However, when used to treat chronic Crohn's Disease in children, the recurrence rate over a 3 year follow up has been reported to be 40%.

A further option is administration of enteral nutrition which is as effective as steroids and can improve nutritional status and growth in children without the side effects commonly associated with drug use.

Many different forms of enteral nutrition have been suggested for use in the treatment or prophylaxis of IBD. In general terms, such nutritional formulations comprise a fat source, free amino acids and/or whole protein and a carbohydrate source as well as vitamins and minerals. Such formulations may be nutritionally complete and may be used as the sole source of nutrition. The formulation is generally a suspension and may, for example, be consumed as a drink or fed to the patient via a nasogastric tube. One example of such a formulation is disclosed in EP-A-0 852 913 (Societe Des Produits Nestle S.A.) and comprises a lipid source providing about 35% to about 50% of energy, a carbohydrate source and casein as whole protein. The lipid profile of this composition is preferably designed to have a weight ratio of n-6:n-3 fatty acids of about 4:1 to about 10:1, most preferably about 6:1 to about 9:1.

There is much published evidence for the protective effects of n-3 polyunsaturated Fatty Acids (PUFAs) in lipids on autoimmune and inflammatory diseases, whilst n-6 PUFAs are associated with an increased incidence and severity of inflammatory diseases. This is a significant finding as the standard western diet contains 10-20 times more n-6 than n-3. Immunosuppressive function of n-3 PUFAs has been shown on T-cell function in both human and animal models of inflammation.

Whilst n-3 PUFAs such as eicosapentaenoic acid (EPA) and docosahexaenoic acid (DHA) have many benefits in diminishing inflammatory responses, as they are the major components of fish oils they are very unpalatable in the diet and susceptible to oxidation leading to rancidity and significant taste problems.

It is therefore an object of the present invention to obviate or mitigate the above mentioned disadvantages.

According to the present invention there is provided a nutritional formulation comprising:
(a) a lipid source,
(b) a carbohydrate source,
(c) a protein source,

wherein the lipid source comprises a weight ratio of n-6:n-3 fatty acids of 1:1 to 3:1, at least 90% by weight of the n-3 fatty acids in the lipid source are provided by α-linolenic acid and at least 90% by weight of the n-6 fatty acids in the lipid source are provided by linoleic acid and components (a), (b) and (c) together provide at least 400 kcal of energy per 100 g total weight of (a)-(c).

The energy content of the formulation as provided by (a), (b) and (c) is calculated on the standard basis that 1 g of protein source and 1 g of carbohydrate source each provide 4 kcal of energy and 1 g of fat source provides 9 kcal of energy.

In accordance with the invention, therefore, the n-3 fatty acids in the lipid source are provided predominately (90% plus) by α-linolenic acid and the n-6 fatty acids are provided predominately (90% plus) by linoleic acid. Additionally, the weight ratio of n-6:n-3 fatty acids is 1:1 to 3:1. These features together provide a number of advantages. In particular, the defined weight ratio (1:1 to 3:1) of n-6 to n-3 fatty acids in conjunction with the fact that these are provided predominately by linoleic acid and α-linolenic acid respectively is important in achieving an anti-inflammatory response. Essential fatty acids such as linoleic and α-linolenic acid are metabolised to product mediators of inflammation known as eicosanoids which as responsible for modulation of the cardiovascular, pulmonary, and secretory systems in addition to the immune system. The levels of these eicosanoids produced are dependent on essential fatty acid intake. The eicosanoids derived from linoleic acid (2 series prostaglandins and 4 series leukotrienes) are more potent inflammatory mediators than those derived from α-linolenic acid (3 series prostaglandins and 5 series leukotrienes) and therefore decreasing the ratio of LA:ALA in the diet is proposed to result in a less inflammatory response. Additionally α-linolenic acid is a precursor of DHA and EPA but may be provided by sources that are much more palatable than those which provide EPA/DHA. As indicated above, the sources of EPA/DHA are generally fish oils that are unpalatable and lead to taste problems. In contrast, α-linolenic acid may be provided by much more palatable sources, as discussed more fully below.

Ideally the weight ratio of n-6:n-3 fatty acids (in the composition defined above) is 1:1 to 3:1, e.g. 1.5:1 to 2:5:1 and ideally about 2:1.

In preferred formulations in accordance with the invention, at least 95% by weight of the n-3 fatty acids in the lipid source is provided by α-linolenic acid. In even more preferred formulations, α-linolenic acid provides at least 98%, more preferably at least 99% and ideally 100% of the n-3 fatty acids in the lipid source. Alternatively or additionally linoleic acid preferably provides at least 95% (more preferably at least 98%, even more preferably 99% and ideally 100% by weight of the n-6 fatty acids in the lipid source.

The lipid source employed in the formulation of the invention may comprise 7.7-14.5% (e.g. 8-12%) by weight of linoleic acid and 2.9-6.8% (e.g. 4-6%) of α-linolenic acid.

Particularly preferred formulations in accordance with the invention comprise a lipid source for which 100% of the n-3 fatty acids are provided by α-linolenic acid and 100% by weight of the n-6 fatty acids are provided by linoleic acid with the weight ratio of linoleic acid to α-linolenic acid being about 2:1

The n-3 and n-6 fatty acids may be provided by a long chain triglyceride oil, e.g. rapeseed, canola, low erucic acid canola, borage, blackcurrant seed, walnut and evening primrose oils. It is most preferred that the n-6 and n-3 fatty acids are provided by rapeseed or canola oil, which as naturally occurring, contains linoleic acid and α-linolenic acid as the sole n-6 and n-3 fatty acids respectively and in a weight ratio of linoleic acid to α-linolenic acid of 2:1. Rapeseed oil contains approximately 20% linoleic acid and 10% α-linolenic acid.

The lipid source will generally also incorporate a medium chain triglyceride oil. Such oils have anti-inflammatory properties and may be selected from fractionated coconut oil, palm kernel oil oils (high in C₈-C₁₂ fatty acids), Miglyol 812™, Miglyol 8108™. The preferred medium chain triglyceride source is fractionated coconut oil.

In preferred formulations in accordance with the invention, the lipid source comprises 45-55% by weight of rapeseed oil and 45-55% by weight of fractionated coconut oil, most preferably about 50% by weight of each.

The protein source preferably comprises at least 95% by weight of whole protein. It is preferred that 100% by weight of the protein source is provided by whole protein. It is however also possible for the whole protein to be used in conjunction with supplementing amino acids. Thus, for example, the protein source may include L-cystine as a supplementing amino acid to increase glutathione levels and increased levels of taurine to act as a free radical scavenger.

It is particularly preferred that the whole protein is provided by a blend of casein and whey protein. Such a blend has advantages with regard to the palatability of the formulation. It is particularly preferred that the protein source comprises, by weight of the protein source, 55-65% by weight casein and 35-45% by weight whey protein. Most preferably the weight ratio of casein:whey is about 60:40.

Further examples of whole proteins that may be used include milk protein, soy protein, rice protein and mixtures thereof.

In a further embodiment of the invention the protein source may be wholly comprised of free amino acids, particularly for the case of patients who cannot tolerate whole protein.

The free amino acids present in the formulation may comprise L-alanine, L-arginine, L-aspartic acid, L-cystine, glycine, L-histidine, L-isoleucine, L-leucine, L-lysine, L-methionine, L-phenylalanine, L-proline, L-serine, L-threonine, L-tryptopahn, L-tyrosine, L-valine, L-carntine and taurine. The nutritional formulation may further comprise L-glutamine as a free amino acid. The presence or absence of glutamine will generally be dictated by the manner in which the nutritional formulation is produced. If heat treatment is required (e.g. for the production of a nutritional formulation in the form of a pasteurised drink) then the use of glutamine will generally be avoided to prevent “off-flavours”.

For a formulation in accordance with the invention to be administered as a liquid (see below) a typical profile for free amino acids (per 100 ml of formulation) would be as follows:

| | |
|---|---|
| L-ALANINE | 0.11 g |
| L-ARGININE | 0.50 g |
| L-ASPARTIC ACID | 0.20 g |
| L-CYSTINE | 0.01 g |
| GLYCINE | 0.18 g |
| L-HISTIDINE | 0.13 g |
| L-ISO LEUCINE | 0.18 g |
| L-LEUCINE | 0.30 g |
| L-LYSINE | 0.22 g |
| L-METHIONINE | 0.14 g |
| L-PROLINE | 0.21 g |
| L-PHENYLALANINE | 0.25 g |
| L-SERINE | 0.13 g |
| L-THREONINE | 0.15 g |
| L-TRYPTOPHAN | 0.06 g |
| L-TYROSINE | 0.05 g |
| L-VALINE | 0.19 g |
| L-CARNITINE | 3.17 mg |
| TAURINE | 6.62 mg |

The carbohydrate source may, for example, be provided by maltodextrin, sucrose, hydrolysed corn starch, glucose polymers or corn syrup solids. Mixtures of these carbohydrate sources may be used. It is preferred that the carbohydrate source is free from lactose. The preferred carbohydrate source is maltodextrin.

As indicated above, the lipid source (a), the carbohydrate source (b) and the protein source (c) together provide at least 400 kcal of energy per 100 g total weight of (a)-(c). The protein source preferably provides 10-15% by weight of the energy with the lipid source providing 30-40% and the carbohydrate source providing 50-60%.

The formulation may be nutritionally complete and therefore include vitamins, minerals and trace elements as required.

One preferred embodiment of the formulation in accordance with the invention comprises 12-18% by weight of the protein source, 15-20% by weight of the fat and 55-60% by weight of the carbohydrate together with vitamins, minerals and trace elements as required. A particularly preferred formulation comprises about 15% by weight of the protein source, about 17.5% of the lipid source and about 58% of the carbohydrate source together with vitamins, minerals and trace elements as required.

The level of vitamins, minerals and trace elements in the formulation may be such as to provide standard recommended levels to a patient when the formulation is consumed at levels appropriate to the energy requirements of the patient concerned. However preferred embodiments of the formulation contain elevated levels of at least one (and preferably a combination of) calcium, phosphorus, zinc, Vitamin C, Vitamin D, Vitamin E, selenium and folacin.

Calcium is preferably present in the formulation and in an amount of 85-105 mg Ca per 100 kcal (of energy provided by (a)-(c), i.e. the lipid source, carbohydrate source and protein source).

The amount of phosphorous in the formulation is preferably in the range 65-85 mg of P per 100 kcal (of energy provided by (a)-(c))

The amounts of calcium and phosphorous quoted in the preceding two paragraphs are advantageous particularly in the case where the formulation is intended for administration to children and adolescents with Inflammatory Bowel Disease since such subjects are at increased risk of impaired bone mineralisation.

Preferably the ratio of Ca:P to formulation is about (1-1.6):1.

The amount of zinc in the formulation is preferably in the range 1.35-1.7 mg of Zn per 100 kcal (of energy provided by (a)-(c). Such levels of zinc are advantageous because up to half of Crohn's patients may be zinc deficient. Furthermore, zinc is a cofactor for the enzymes that convert linoleic and α-linolenic acids to their longer chain metabolites.

The formulation preferably contains Vitamin C in an amount of 15-35 mg per 100 kcal (of energy provided by (a)-(c)). This level of Vitamin C is advantageous for a number of reasons. Firstly, it has been reported that oxidant stress from inflamed mucosa in the gut contributes to a 35% loss of ascorbate in Crohn's patients. Secondly, paediatric Crohns patients have been shown to have approximately half the circulating plasma Vitamin C concentrations of age matched healthy control children. Thirdly, Vitamin C also converts oxidised Vitamin E back to its antioxidant form, thus providing a protective effect on Vitamin E levels. Fourthly, studies have shown an overproduction of reactive oxygen species within the inflamed intestine and colon in patients suffering from Inflammatory Bowel Disease. Therefore, the provision of Vitamin C as an aqueous phase antioxidant is beneficial in the aqueous environment.

Vitamin E is preferably present in the formulation and in amount of 5-9 mg per gram of PUFAs in the formulation. This level of Vitamin E is advantageous in allowing for increased oxidative stress in Crohn's Disease and thus protect lipids against peroxidation.

Vitamin D is preferably present in the formulation in an amount of 0.75-1.15 μg per 100 kcal (of energy provided by (a)-(c)). This Vitamin D level is advantageous to counter Vitamin D deficiency that has been reported in 27% of patients with Crohn's Disease.

Selenium is preferably present in the formulation in an amount of 7.5-10.5 μg per 100 kcal (of energy provided by (a)-(c)). This level is advantageous since patients suffering from Crohn's Disease have decreased levels of selenium which is an essential cofactor for glutathione peroxidise (an antioxidant enzyme). As such, Crohns patients have a low glutathione peroxidise activity.

Folic acid is preferably present in the formulation in an amount of 30-41.5 μg per 100 kcal (of energy provided by (a)-(c)) to supplement low plasma folate levels observed in Crohn's patients.

Flavourings may be incorporated in the formulation as desired.

Formulations in accordance with the invention may be prepared and supplied as powders (e.g. in sachets or other moisture proof container) and subsequently admixed with water for the purposes of administration to the patient. Typically, the formulation will be diluted so as to provide about 1.0-1.7 kcal/ml.

The diluted formulation may be consumed by the patient as a drink or fed to the patient via a nasogastric tube.

The formulation may be used for treatment (including prophylaxis) of a number of inflammatory conditions of the gastrointestinal tract, e.g. Inflammatory Bowel Disease or gastrointestinal inflammation associated with cystic fibrosis. The formulation is particularly useful for the treatment (including prophylaxis) of Crohn's Disease.

The formulation may be used for treating acute (i.e. active) disease (particularly paediatric Crohn's Disease), in which case it will be the sole source of nutrition and nutrionally complete.

The formulation may also be used for the maintenance of remission from disease in which case it will be nutrionally complete but used as a supplement in addition to everyday foods.

The invention is illustrated by reference to the following non-limiting Examples.

### EXAMPLE 1

A powdered formulation in accordance with the invention which is suitable for dilution with water to form an enteral feed comprises:

| Component | Per 100 g Powder | |
|---|---|---|
| Maltodextrin | 58 | g |
| Casein | 9 | g |
| Whey Protein | 6 | g |
| Rapeseed Oil | 8.75 | g |
| Fractionated Coconut Oil | 8.75 | g |
| Vitamin A | 310 | μg |
| Vitamin C | 72 | mg |
| Vitamin D | 3.5 | μg |
| Vitamin E | 12.2 | μg |
| Calcium | 428 | mg |
| Phosphorous | 338 | mg |
| Selenium | 36 | μg |

| | |
|---|---|
| Other Vitamins/Minerals/Trace Elements | Balance |

The above formulation provides about 450 kcal of energy per 100 g powder.

The formulation may be diluted with water to provide a 33.3% w/v suspension providing an energy density of 1.5 kcal/ml.

### EXAMPLE 2

Below is an example of a feeding regime (either oral/nasogastric administration) for a male Crohn's disease patient (aged 15 years; weight 50 kg) demonstrating how the energy requirements can be met by the powdered formulation of Example 1.

Energy requirements: Range from 2074-2755 kcal/day based on Schofield Equation and EAR. Therefore aim for 2000-2750 kcal/day. In view of the likelihood of a Crohn's disease patient being underweight it is aimed to feed 2250 kcal/day.

Protein requirements: 55.2-62.5 g/day based on RNI and PENG (Parenteral & Enteral Nutrition Group of dietitians recommendations).

Fluid Requirements:
100 ml/kg for first 10 kg=1000 ml
50 ml/kg for next 10 kg=500 ml
25 ml/kg for rest of weight (30 kg)=750 ml
Total=2250 ml.

On starting feeding this nutritional composition, the recommended volume can be built up over 2-3 days—this feeding regime must be developed by a dietitian to ensure that adequate fluid intake is achieved.

Tube Feeding Regime (Via a Feeding Pump)—Build Up Regime

Day 1
300 g powder to final volume of 1200 ml at 60 ml/h for 20 hours
500 ml water via pump at 125 ml/h and 100 ml water flush before and after feed.
Provides: 1350 kcal, 45 g protein, 1900 ml fluid

Day 2
500 g powder made up to 2000 ml at 100 ml/h for 20 hours
150 ml water flush before and after feed.
Provides: 2250 kcal, 75 g protein and 2300 ml fluid

Day 3
500 g powder made up to 1700 ml at 125 ml/h for 14 hours
200 ml flush before/after feed and 1×200 ml water bolus in rest period.
Provides: 2250 kcal, 75 g protein and 2300 ml fluid

Day 4 and Continued
500 g powder made up to 1500 ml at 125 ml/h for 12 hours
250 ml water before and after feed and 1×250 ml water bolus in rest period.
Provides 2250 kcal, 75 g protein and 2250 ml fluid.

Oral Regime: Build Up Regime

Day 1
50 g powder made up to 200 ml×6 glassess
At least 1000 ml additional water as drinks in day

Day 2
75 g powder made up to 250 ml×6 glasses a day
Plus additional 750 ml water

Day 3
100 g powder made up to a 300 ml volume×5 a day
Plus an additional 750 ml water.

### EXAMPLE 3

This Example reports the results of a study on the efficacy of the 33.3% w/v suspension of the formulation described in Example 1 for the treatment of paediatric patients with Crohn's Disease.

Paediatric patients aged 5-16 years on first presentation of acute Crohn's Disease (n=14) received the suspension (of the formulation of Example 1) containing the anti-inflammatory fatty acid blend as their sole source of nutrition for six weeks. Plasma fatty acid profiles were investigated from samples taken at baseline and after 6 weeks on nutritional intervention.

The results are shown are shown in the following Table.

| Nutritional Intervention (n = 14) | | |
|---|---|---|
| | Fractional concentration (%) | Paired t-test |

| | Start | End | P |
|---|---|---|---|
| 14:0 | 0.7 ± 0.4 | 1.1 ± 0.7 | |
| 16:0 | 25.2 ± 2.7 | 25.2 ± 4.7 | |
| 16:1n-7 | 2.5 ± 1.2 | 2.6 ± 0.9 | |
| 18:1n-9 | 28.4 ± 3.0 | 26.2 ± 2.2 | |
| 18:2n-6 | 20.7 ± 4.3 | 22.2 ± 4.9 | 0.031 |
| 18:3n-6 | 0.3 ± 0.1 | 0.4 ± 0.2 | |
| 18:3n-3 | 0.7 ± 0.4 | 1.2 ± 0.6 | 0.016 |
| 20:0 | 0.2 ± 0.1 | 0.2 ± 0.1 | |
| 22:0 | 0.2 ± 0.1 | 0.3 ± 0.1 | 0.003 |
| 20:3n-6 | 1.6 ± 0.5 | 2.0 ± 0.7 | 0.010 |
| 20:4n-6 | 5.7 ± 1.8 | 4.1 ± 1.5 | <0.0001 |
| 20:5n-3 | 0.6 ± 0.2 | 0.9 ± 0.4 | 0.009 |
| 24:0 | 0.3 ± 0.1 | 0.2 ± 0.1 | |
| 22:5n-3 | 0.6 ± 0.2 | 0.7 ± 0.3 | |
| 22:6n-3 | 1.5 ± 0.7 | 1.2 ± 0.8 | |

Values are mean ± SD

The above results demonstrate that in the patients receiving the nutritional intervention, there was a significant increase in 18:2n-6, 18:3n-3, 22:0, 20:3n-6 and 20:5n-3 between the start and end of the study. 20:4n-6 concentration decreased between the start and end of the study.

Absolute changes between levels of fatty acids at the start and end of the study were fairly small. However the data presented above demonstrates that use of a nutritional intervention comprising the anti-inflammatory fatty acid blend results in an effect on blood fatty acid composition with the biological effect of reduction in 20:4n-6 levels and the almost reciprocal increase in 20:5n-3 levels thus modulating the (n-6):(n-3) ratio.

1. A nutritional formulation comprising:
(a) a lipid source,
(b) a carbohydrate source, and
(c) a protein source, and
(d) calcium in an amount of 85-105 mg Ca and phosphorous in an amount of 65-85 mg of P per 100 kcal of energy provided by (a)-(c).
wherein the lipid source comprises a weight ratio of n-6:n-3 fatty acids of 1:1 to 3:1, at least 90% by weight of the n-3 fatty acids in the lipid source are provided by α-linolenic acid and at least 90% by weight of the n-6 fatty acids in the lipid source are provided by linoleic acid and components (a), (b) and (c) together provide at least 400 kcal of energy per 100 g total weight of (a)-(c).

2. A formulation as claimed in claim 1 wherein the protein provides 10-15% of said energy, the fat provides 30-40% and the carbohydrate provides 50-60%.

3. A formulation as claimed in claim 1 which, based on the total weight of (a)-(c), comprises 12-18% by weight of the protein, 15-20% by weight of the fat and 55-60% by weight of the carbohydrate.

4. A formulation as claimed in claim 1; wherein the rate of n-6:n-3 fatty acids is 1.5:1 to 2.5:1.

5. A formulation as claimed in claim 1 wherein at least 95% by weight of the n-3 fatty acids in the lipid source is provided by α-linolenic acid.

6. A formulation as claimed in claim 1 wherein at least 95% by weight of the n-6 fatty acids in the lipid source is provided by linoleic acid.

7. A formulation as claimed in claim 1 wherein at least 95% by weight of the protein source is provided by whole protein.

8. A formulation as claimed in claim 7 wherein the protein source comprises, by weight of the protein source, 55%-65% by weight casein and 35%-45% by weight whey protein.

9. A formulation as claimed in claim 1 wherein the protein source comprises free amino acids.

10. A formulation as claimed in claim 9 wherein the free amino acids comprise: L-alanine, L-arginine, L-aspartic acid, L-cystine, glycine, L-histidine, L-isoleucine, L-leucine, L-lysine, L-methionine, L-phenylalanine, L-proline, L-serine, L-threonine, L-tryptophan, L-tyrosine, L-valine, L-carnitine and taurine.

11. A formulation according to claim 1 for the treatment of inflammatory conditions of the gastrointestinal tract.

12. A formulation according to claim 1 for the treatment of impaired bone mineralization in patients with inflammatory bowel disease.

13. A method of treating an inflammatory condition of the gastrointestinal tract comprising administrating to a patient in need of such treatment a formulation as claimed in claim 1.

14. A method as claimed in claim 13 wherein the inflammatory condition is Inflammatory Bowel Disease.

15. A method as claimed in claim 13 wherein the Inflammatory Bowel Disease is Crohn's Disease.

16. A method as claimed in claim 13 wherein the inflammatory condition is one associated with cystic fibrosis.
